(19) United States
(12) Patent Application Publication
(10) Pub. No.: US 2021/0228195 A1
(43) Pub. Date: Jul. 29, 2021

(54) FLUTE CATHETER AND METHODS OF USING THE SAME

(71) Applicant: Ghassan S. Kassab, La Jolla, CA (US)

(72) Inventor: Ghassan S. Kassab, La Jolla, CA (US)

(21) Appl. No.: 17/157,211

(22) Filed: Jan. 25, 2021

Related U.S. Application Data
(60) Provisional application No. 62/965,156, filed on Jan. 23, 2020.

(51) Int. Cl. A61B 17/00

(52) U.S. Cl. CPC A61B 17/00234; A61B 2017/306

(57) ABSTRACT

A catheter for the gentle opening of luminal valves and method of use is disclosed. The catheter comprises a suction lumen and a guidewire within. The suction lumen provides an atraumatic amount of suction force to draw valve leaflets open whereupon the guidewire can be advanced through the now open valve or the contents of the luminal organ beyond the valve can be aspirated into the device. When the guidewire is advanced, the catheter can follow the guide wire beyond the opened valve, thereby allowing retrograde access of luminal organs beyond normally closed valves. The process can be repeated to open a second valve located upstream of a first valve.

### PRIORITY

The present patent application is related to, and claims the priority benefit of, U.S. Provisional Patent Application Ser. No. 62/965,156, filed on Jan. 23, 2020, the contents of which are hereby incorporated by reference in their entirety into this disclosure.

### BACKGROUND

Intraluminal procedures are minimally invasive to patients and result in less risk and quicker healing times overall when compared to traditional open surgery. Some intraluminal procedures can be performed in an antegrade or retrograde direction. Certain patients may not be good candidates for a procedure using an antegrade approach. In some cases, the anatomy may not be favorable to an antegrade approach. The procedure may be more difficult due to convoluted anatomy and therefore take longer to complete which results in increased risk to the patient. In such instances, a retrograde approach may be preferred.

Valve structures and/or thin wall organs can complicate intraluminal procedures. Special care must to not perforate the walls or damage valve structures. The thoracic duct of the lymphatic system is one such structure. The duct comprises multiple valves and is relatively thin walled.

Thus there is a need for a device that can safely traverse valved structures in a retrograde direction to access treatment sites without causing damage to the organ.

### BRIEF SUMMARY

The present disclosure describes an embodiment of an intraluminal catheter capable of safe retrograde access of a valved luminal organ.

In one embodiment of the present disclosure, the device comprises a catheter having a distal end, the distal end having a suction lumen and an expandable and collapsible distal portion, wherein the expandable and collapsible distal portion expands against the organ wall and valve structure to support the organ wall from collapse when suction is applied through the suction lumen. In a further embodiment the device further comprises a guidewire.

An exemplary embodiment of the present disclosure comprises an intraluminal catheter comprising: a distal end, wherein the distal end comprises an expandable and collapsible portion; and a suction lumen ending at the distal end.

An exemplary embodiment of the present disclosure comprises an intraluminal catheter comprising: a distal end, wherein the distal end comprises an expandable and collapsible portion; a suction lumen ending at the distal end; and a distal tip, wherein the expandable and collapsible portion extends from the distal tip and terminates at a distance proximal of the distal tip.

An exemplary embodiment of the present disclosure comprises an intraluminal catheter comprising: a distal end, wherein the distal end comprises an expandable and collapsible portion; a suction lumen ending at the distal end; and wherein the suction lumen provides 20 mmHG to 50 mmHG of suction.

An exemplary embodiment of the present disclosure comprises an intraluminal catheter comprising: a distal end, wherein the distal end comprises an expandable and collapsible portion; a suction lumen ending at the distal end; and wherein the expandable and collapsible portion is expandable to rest against a luminal organ walls and against a valve.

An exemplary embodiment of the present disclosure comprises an intraluminal catheter comprising: a distal end, wherein the distal end comprises an expandable and collapsible portion; a suction lumen ending at the distal end; wherein the suction lumen provides 20 mmHG to 50 mmHG of suction; and the expandable and collapsible portion is resistant to the 20 mmHG to 50 mmHG of suction.

In an exemplary embodiment, a method of using an intraluminal catheter the method comprises the steps of: advancing a catheter in a retrograde direction within a luminal organ toward a valve; expanding an expandable and collapsible portion of the catheter against the luminal organ walls and valve; and applying suction to draw open the valve.

In an exemplary embodiment, a method of using an intraluminal catheter the method comprises the steps of: advancing a catheter in a retrograde direction within a luminal organ toward a valve; expanding a expandable and collapsible portion of the catheter against the luminal organ walls and valve; and applying suction to draw open the valve; advancing a guidewire beyond the open valve; collapsing the expandable and collapsible portion; and advancing the catheter over the guidewire to a second position.

In an exemplary embodiment, a method of using an intraluminal catheter the method comprises the steps of: advancing a catheter in a retrograde direction within a luminal organ toward a valve; expanding a expandable and collapsible portion of the catheter against the luminal organ walls and valve; and applying suction to draw open the valve; applying suction to aspirate the contents of the luminal organ.

In an exemplary embodiment, a method of using an intraluminal catheter the method comprises the steps of: advancing a catheter in a retrograde direction within a luminal organ toward a valve; expanding a expandable and collapsible portion of the catheter against the luminal organ walls and valve; and applying suction to draw open the valve; applying suction to aspirate the contents of the luminal organ; advancing a guidewire beyond the open valve; collapsing the expandable and collapsible portion; and advancing the catheter over the guidewire to a second position.

In an exemplary embodiment, an intraluminal catheter comprises a distal end, wherein the distal end comprises an expandable and collapsible portion and a suction lumen ending at the distal end. The catheter may also comprise a distal tip, wherein the expandable and collapsible portion extends from the distal tip and terminates at a distance proximal of the distal tip.

In an exemplary embodiment of an intraluminal catheter, in an expanded configuration the expandable and collapsible portion is tapered. In another embodiment of an intraluminal catheter, in an expanded configuration a proximal edge of the expandable and collapsible portion is perpendicular to the catheter

In an exemplary embodiment of an intraluminal catheter, the suction lumen is configured to provide 20 mmHG to 50 mmHG of suction. In an exemplary embodiment of an intraluminal catheter, the expandable and collapsible portion is resistant to the 20 mmHG to 50 mmHG of suction.

In an exemplary embodiment, the expandable and collapsible portion is expandable to abut against a luminal organ walls and against a valve structure.

In an embodiment of a method of using an intraluminal catheter, the method comprises the steps of: advancing a catheter in a retrograde direction within a luminal organ toward a valve; expanding an expandable and collapsible portion of the catheter; and applying suction to draw open the valve.

In a further embodiment, the method of using the intraluminal catheter comprises the step of advancing the catheter so as to rest against a valve structure. In a further embodiment, the method of using the intraluminal catheter comprises the step of expanding the expandable and collapsible portion of the catheter to abut the luminal organ walls.

In an exemplary embodiment of a method of using an intraluminal catheter, the step of applying suction to draw open a valve comprises the step of 20 mmHG to 50 mmHG of suction.

In an exemplary embodiment of a method of using the intraluminal catheter, the method comprises the step of applying suction to aspirate the contents of the luminal organ.

In an further embodiment of a method of using an intraluminal catheter the method of using the intraluminal catheter of claim 8 further comprises the steps of: advancing a guidewire beyond the a first open valve; collapsing the expandable and collapsible portion; and advancing the catheter over the guidewire to a second position wherein the second position is near a second valve; expanding an expandable and collapsible portion of the catheter; and applying suction to draw open the second valve.

In an exemplary embodiment of a method of using an intraluminal catheter, the method comprises the steps of advancing a catheter in a retrograde direction within a luminal organ toward a first valve so as to rest against a first valve structure; expanding an expandable and collapsible portion of the catheter to abut the luminal organ walls; applying suction to draw open the first valve; advancing a guidewire beyond the open first valve; collapsing the expandable and collapsible portion; advancing the catheter over the guidewire beyond the open first valve, to a second position wherein the second position is near a second valve; expanding for a second time an expandable and collapsible portion of the catheter; and applying suction to draw open the second valve.

In an exemplary embodiment of a method of using an intraluminal catheter, the steps of applying suction to draw open the first valve and applying suction to draw open the second valve further comprise the step of applying 20 mmHG to 50 mmHG of suction.

In an exemplary embodiment of a method of using an intraluminal catheter, the step of advancing the catheter over the guidewire beyond the open first valve, to a second position wherein the second position is near a second valve further comprises the step of advancing the catheter so as to rest against a second valve structure.

In an exemplary embodiment of a method of using an intraluminal catheter, the step of expanding for a second time an expandable and collapsible portion of the catheter further comprises the step of expanding the expandable and collapsible portion of the catheter to abut the luminal organ walls.

In an exemplary embodiment of a method of using an intraluminal catheter, after applying suction to draw open the second valve, a step of applying suction to aspirate the contents of the luminal organ is performed.

In an exemplary embodiment of a method of using an intraluminal catheter, the method further comprises the step of applying further suction to aspirate the contents of the luminal organ, after the step of applying suction to draw open the second valve.

### BRIEF DESCRIPTION OF THE DRAWINGS

The disclosed embodiments and other features, advantages, and disclosures contained herein, and the matter of attaining them, will become apparent and the present disclosure will be better understood by reference to the following description of various exemplary embodiments of the present disclosure taken in conjunction with the accompanying drawings, wherein:

FIG. 1 shows a side view of a luminal organ having two valves, according to an exemplary embodiment of the present disclosure;

FIG. 2 shows an expanded flute catheter deployed in the luminal organ of FIG. 1, according to an exemplary embodiment of the present disclosure;

FIG. 3 shows a flute catheter applying suction to the luminal organ of FIG. 1, according to an exemplary embodiment of the present disclosure;

FIG. 4 shows a flute catheter and guidewire advanced through the luminal organ of FIG. 1, according to an exemplary embodiment of the present disclosure; and

FIG. 5 shows two exemplary embodiments of the flute portion.

An overview of the features, functions and/or configurations of the components depicted in the various figures will now be presented. It should be appreciated that not all of the features of the components of the figures are necessarily described. Some of these non-discussed features, such as various couplers, etc., as well as discussed features are inherent from the figures themselves. Other non-discussed features may be inherent in component geometry and/or configuration.

### DETAILED DESCRIPTION

For the purposes of promoting an understanding of the principles of the present disclosure, reference will now be made to the embodiments illustrated in the drawings, and specific language will be used to describe the same. It will nevertheless be understood that no limitation of the scope of this disclosure is thereby intended.

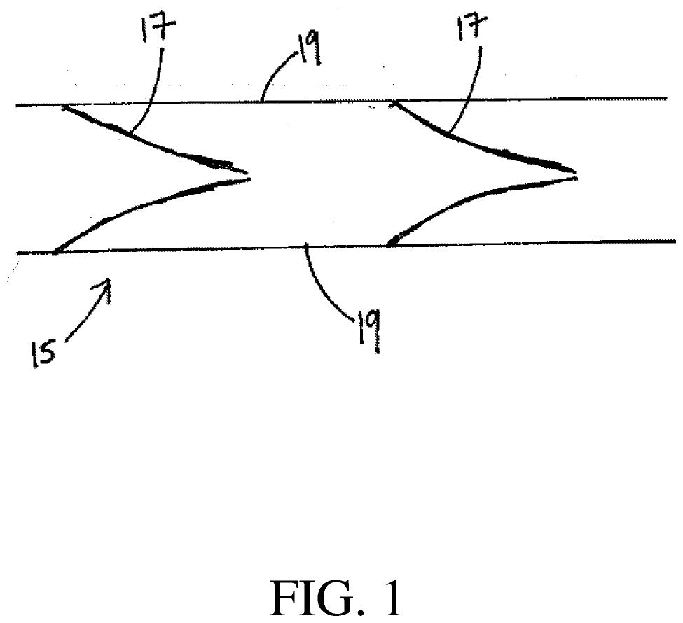

FIG. 1 shows a luminal organ 15 having two valves 17. For illustrative purposes an embodiment of the present disclosure will be described as deployed in the luminal organ 15 of FIG. 1.

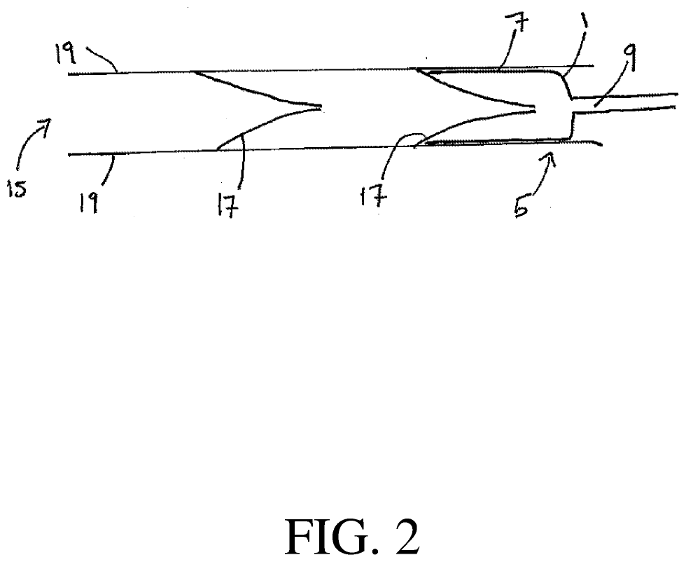

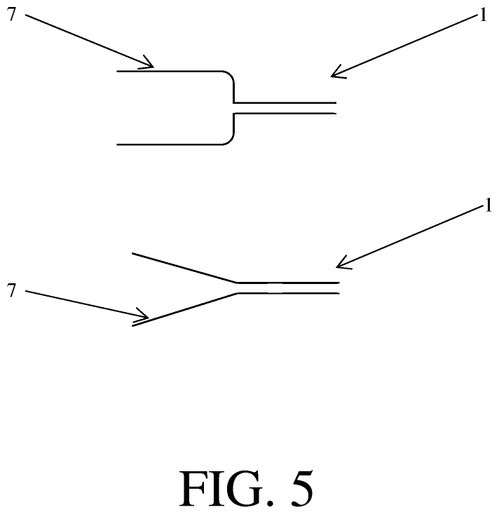

The distal end of an exemplary device for safe retrograde access of a structure with valves 17 of the present disclosure is shown in FIGS. 2-4. As shown in FIGS. 2-4, an exemplary device comprises an intraluminal catheter 1. The distal end 5 of the intraluminal catheter 1 (also referred to as a flute catheter 1) has an expandable and collapsible portion 7 (also referred to as a flute 7), and a suction lumen 9 which provides suction at the distal end 5 of the catheter 1. The flute portion 7 comprises the exterior of the catheter 1 and extends proximally from the distal tip for a distance. The flute portion 7 comprises a depth such that the valve leaflets 17 can be contained within the flute 7. In this embodiment, the flute portion 7 ends proximally in an edge perpendicular to the catheter 1, but may be tapered depending on the construction of the particular embodiment as shown in FIG. 5. The flute 7 is sized such that on expansion fits or abuts or otherwise rests against the luminal organ walls 19 to provide support in the presence of suction.

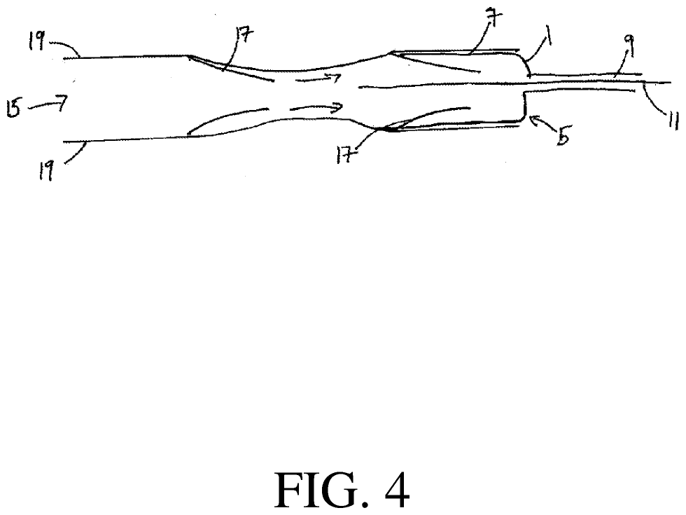

A guidewire 13 is slidably engaged with the catheter 1 and may share the same lumen as the suction lumen 9 as in FIG. 4 or have a separate guidewire lumen in the catheter. It is understood that the catheter 1 comprises a proximal end (not shown) from where an operator can access and manipulate the catheter 1.

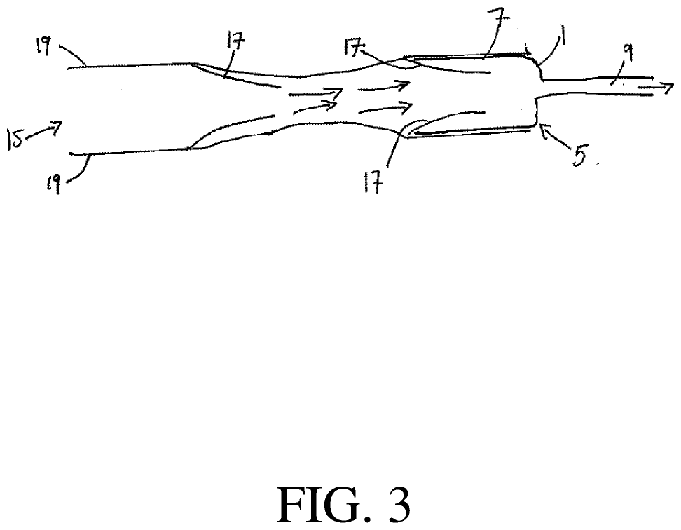

FIG. 2 shows a deployed and expanded flute 7. The deployed flute 7 abuts the walls 19 of the luminal organ 15 and against the valve structure 17. As shown in FIG. 3, suction introduced through the suction lumen 9 provides enough suction to open the valves, around 20 mmHG to 50 mmHG. The directional arrows of FIGS. 3-4 indicate suction. Applying suction will draw the flute 7 engaged valve 17 open, while supporting the organ structure 15 at the level of valve leaflet 17 attachment. Where a second valve 17 is close enough or the suction is strong enough, the second valve 17 may also be slightly engaged open by the suction such as in FIG. 3. The expanded flute 7 is sufficiently stiff to resist the introduced suction and prevent the organ walls 19 from collapsing from the applied suction.

At this stage, the suction can be applied gradually to aspirate luminal organ 15 contents, such as lymphatic fluid in the case of the thoracic duct, as shown in FIG. 3. The contents can be collected for diagnosis (e.g. immunological biomarkers, lipid disorders, etc) and therapy (e.g., CAR T-cell therapy for cancer treatment, to decompress the lymphatic system in heart failure, to remove lipid cells for weight control, etc.).

Where access is required more distally, such as in a vein, the guidewire 13 can be advanced through the suction engaged valve 17 as shown in FIG. 4. Then the flute 7 can be collapsed and the catheter 1 advanced over the guidewire 13 to reach the next valve 17. Then the expansion, suction and advancement process as described in FIG. 2-4 can be repeated as needed to advance through subsequent valves 17.

An exemplary method of use comprises the steps of advancing a catheter 1 in a retrograde direction within a luminal organ 15 toward a valve 17; expanding the expandable and collapsible portion 7 against the luminal organ walls 19 and valve 17; and applying suction to draw open the valve 17.

In a further exemplary method of use, after the applied suction draws open the valve 17, further suction can be applied to aspirate the contents of the luminal organ 15.

In a further exemplary method of use, after the applied suction draws open the valve 17, a guidewire can be advanced through the valve 17. Then the expandable and collapsible portion 7 can be collapsed and the catheter 1 advanced over the guidewire 13 to a second position. At a second position the expandable and collapsible portion 7 can be expanded again against a second valve 17 and the luminal organ wall 19, and suction can be applied again to draw open the second valve 17.

While various embodiments of devices for safe retrograde access of valved structures and methods for using the same have been described in considerable detail herein, the embodiments are merely offered as non-limiting examples of the disclosure described herein. It will therefore be understood that various changes and modifications may be made, and equivalents may be substituted for elements thereof, without departing from the scope of the present disclosure. The present disclosure is not intended to be exhaustive or limiting with respect to the content thereof.

Further, in describing representative embodiments, the present disclosure may have presented a method and/or a process as a particular sequence of steps. However, to the extent that the method or process does not rely on the particular order of steps set forth therein, the method or process should not be limited to the particular sequence of steps described, as other sequences of steps may be possible. Therefore, the particular order of the steps disclosed herein should not be construed as limitations of the present disclosure. In addition, disclosure directed to a method and/or process should not be limited to the performance of their steps in the order written. Such sequences may be varied and still remain within the scope of the present disclosure.

1. An intraluminal catheter comprising:
a distal end, wherein the distal end comprises an expandable and collapsible portion
a suction lumen ending at the distal end.

2. The device of claim 1, further comprising:
a distal tip,
wherein the expandable and collapsible portion extends from the distal tip and terminates at a distance proximal of the distal tip.

3. The device of claim 2 wherein in an expanded configuration the expandable and collapsible portion is tapered.

4. The device of claim 2 wherein in an expanded configuration a proximal edge of the expandable and collapsible portion is perpendicular to the catheter

5. The device of claim 1, wherein the suction lumen is configured to provide 20 mmHG to 50 mmHG of suction.

6. The device of claim 5, wherein the expandable and collapsible portion is resistant to the 20 mmHG to 50 mmHG of suction.

7. The device of claim 1, wherein the expandable and collapsible portion is expandable to abut against a luminal organ walls and against a valve structure.

8. A method of using an intraluminal catheter, the method comprising the steps of:
advancing a catheter in a retrograde direction within a luminal organ toward a valve;
expanding an expandable and collapsible portion of the catheter; and
applying suction to draw open the valve.

9. The method of using the intraluminal catheter of claim 8 wherein the step of advancing a catheter in a retrograde direction within a luminal organ toward a valve further comprises the step of advancing the catheter so as to rest against a valve structure.

10. The method of using the intraluminal catheter of claim 8 wherein the step of expanding an expandable and collapsible portion of the catheter further comprises the step of expanding the expandable and collapsible portion of the catheter to abut the luminal organ walls.

11. The method of using the intraluminal catheter of claim 8 wherein the step of applying suction to draw open the valve further comprises the step of 20 mmHG to 50 mmHG of suction.

12. The method of using the intraluminal catheter of claim 8 further comprising the steps of:
applying suction to aspirate the contents of the luminal organ.

13. The method of using the intraluminal catheter of claim 8 further comprising the steps of:
advancing a guidewire beyond the open valve;
collapsing the expandable and collapsible portion; and
advancing the catheter over the guidewire to a second position wherein the second position is near a second valve.

14. The method of using the intraluminal catheter of claim 13 further comprising the steps of:
expanding an expandable and collapsible portion of the catheter; and
applying suction to draw open the second valve.

15. A method of using an intraluminal catheter, the method comprising the steps of:
advancing a catheter in a retrograde direction within a luminal organ toward a first valve so as to rest against a first valve structure;
expanding an expandable and collapsible portion of the catheter to abut the luminal organ walls;
applying suction to draw open the first valve;
advancing a guidewire beyond the open first valve;
collapsing the expandable and collapsible portion;
advancing the catheter over the guidewire beyond the open first valve, to a second position wherein the second position is near a second valve;
expanding for a second time an expandable and collapsible portion of the catheter; and
applying suction to draw open the second valve.

16. The method of using the intraluminal catheter of claim 15 wherein the steps of applying suction to draw open the first valve and applying suction to draw open the second valve further comprise the step of applying 20 mmHG to 50 mmHG of suction.

17. The method of using the intraluminal catheter of claim 15 wherein the step of advancing the catheter over the guidewire beyond the open first valve, to a second position wherein the second position is near a second valve further comprises the step of advancing the catheter so as to rest against a second valve structure.

18. The method of using the intraluminal catheter of claim 15 wherein the step of expanding for a second time an expandable and collapsible portion of the catheter further comprises the step of expanding the expandable and collapsible portion of the catheter to abut the luminal organ walls.

19. The method of using the intraluminal catheter of claim 15 wherein after applying suction to draw open the second valve, as step of applying suction to aspirate the contents of the luminal organ is performed.

20. The method of using the intraluminal catheter of claim 15 further comprising the step of applying further suction to aspirate the contents of the luminal organ, after the step of applying suction to draw open the second valve.
